(19) United States
(12) Patent Application Publication
(10) Pub. No.: US 2012/0237620 A1
(43) Pub. Date: Sep. 20, 2012

(54) Hot Flash Relief Compositions and Methods

(75) Inventor: Jeanne Raede, Thousand Oaks, CA (US)

(73) Assignee: Beaumont Products, Inc., Kennesaw, GA (US)

(21) Appl. No.: 13/422,441

(22) Filed: Mar. 16, 2012

Related U.S. Application Data
(60) Provisional application No. 61/453,257, filed on Mar. 16, 2011.

(51) Int. Cl. A61K 31/045; A61P 43/00; A61K 36/534

(52) U.S. Cl. 424/747; 514/729

(57) ABSTRACT

Topical compositions and methods for treating hot flashes and night sweats are disclosed. As disclosed herein, a menthol-based composition is topically sprayed onto the back of the neck in order to alleviate symptoms associated with hot flashes and night sweats.

### CROSS REFERENCE

This is a division of U.S. Provisional Application Ser. No. 61/453,257, filed Mar. 16, 2011.

### BACKGROUND

The present disclosure generally relates to hot flashes. More particularly, the present disclosure provides a composition and method for treating, reducing, or ameliorating the effects of hot flashes.

There are three generally accepted categories of menopause: perimenopause, menopause, and induced menopause. Menopause is defined as the last and final menstrual period and is usually confirmed when a woman misses her period for twelve consecutive months. Menopause itself is associated with a reduced functioning of the ovaries due to natural aging of the body. This reduction in function of the ovaries leads to lower levels of estrogen and other natural hormones. This phase marks the permanent end of fertility for women. Symptoms of menopause include hot flashes, night sweats, sleep disturbances, vaginal dryness, mood swings, headaches, and irritability.

Perimenopausal symptoms can begin years ahead of actual menopause, or the final menstrual cycle. It is considered a transition phase and can often last up to six years or more. Symptoms of perimenopause are much like menopausal symptoms.

Induced menopause occurs when menstrual periods stop due to medical intervention, such as surgical removal of both ovaries, or cancer treatments. Hot flashes and night sweats due to surgical procedures or cancer treatments can affect both men and women. Symptoms for induced menopause are often intensified due to the absence of the perimenopausal transition.

Researchers believe that hot flashes occur when the hypothalamus nerve cells in the brain, which help to regulate body temperature, mistakenly sense that the body is too warm and attempt to cool it down. The hypothalamus cells have estrogen receptors, so that when the estrogen levels decline due to perimenopause, menopause, or induced menopause, the internal temperature regulators of the body do not function as well, resulting in a hot flash or night sweat.

The body then responds to the signals generated by the hypothalamus nerve cells in the brain by increasing heart rate and blood flow to the extremities. As skin temperature rises due to the increased heart rate and blood flow, the heat and the sweating mechanism begins. Once the body temperature drops from the cooling effect, blood vessels constrict and the individual may begin to feel cold and clammy. When this occurs at night during sleep, it is considered a “night sweat” and often results in sleep disruption, fatigue, and irritability.

Every woman is different, but hot flashes can occur during any of the three categories of menopause in varying degrees of intensity. Triggers for hot flashes are different for each woman, and can include stress, anxiety, certain foods, caffeine, hot drinks, alcohol, hot rooms, or no discernable trigger whatsoever other than the body's internal hormonal and chemical fluctuations. Night sweats are also very common in men and well who have gone through major surgeries or cancer treatments. Night sweats have also been linked as the secondary effect of certain medications.

There are known medications, including hormone therapy, for treating menopause and related hot flash symptoms. However, such medication must be ingested and often has side effects. Accordingly, there is a continuing need for a composition and method for treating hot flashes which is quickly effective and does not have any adverse side effects.

### DETAILED DESCRIPTION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of medicine, organic chemistry, biochemistry, molecular biology, pharmacology, and the like, which are within the skill of the art. Such techniques are explained fully in the literature.

It must be noted that, as used in the specification and the appended claims, the singular forms “a,” “an,” and “the” include plural referents unless the context clearly dictates otherwise. Thus, for example, reference to “a surfactant” includes a plurality of surfactants. In this specification and in the claims that follow, reference will be made to a number of terms that shall be defined to have the following meanings unless a contrary intention is apparent.

As used herein, the following terms have the meanings ascribed to them unless specified otherwise. In this disclosure, “comprises,” “comprising,” “containing,” and “having” and the like can have the meaning ascribed to them in U.S. Patent law and can mean “ includes,” “including,” and the like; “consisting essentially” of or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments.

As used herein, the following phrase, “at least one of the following: X, Y, and Z” (where X, Y, and Z can represent composition ingredients or components), means any one of X, Y, or Z, or any combination thereof. Fox example, the phrase “at least one of the following: X, Y, and Z” can mean and one of X alone, Y alone, Z alone, or combinations of X and Y, X and Z, or Y and Z, etc.

Prior to describing the various embodiments, the following definitions are provided and should be used unless otherwise indicated.

### Definitions:

The term “derivative” refers to a modification to the disclosed compounds.

As used herein the terms “treat,” “treating,” or “treatment” of a condition includes preventing the condition from occurring in a recipient host that may be predisposed to the condition but does not yet experience or exhibit symptoms of the condition (prophylactic treatment), inhibiting the condition (slowing or arresting its development), relieving the condition (causing regression of the condition), and/or preventing recurrence or relapse of the condition. In the context of the present disclosure, the term may also refer to generating a physiological or psychological state that results in aversion to, and thereby, reduced acceptance of, a stimulant.

The compounds of the present disclosure may be administered in the form of a pharmaceutically acceptable salt. The term “pharmaceutically acceptable salt” refers to salts prepared from pharmaceutically acceptable non-toxic bases or acids including inorganic or organic bases and inorganic or organic acids. Salts of basic compounds encompassed within the term “pharmaceutically acceptable salt” refer to non-toxic salts of the compounds of this disclosure may be generally prepared by reacting a free base with a suitable organic or inorganic acid. Representative salts of basic compounds of the present disclosure include, but are not limited to, the following: acetate, benzenesulfonate, benzoate, bicarbonate, bisulfate, bitartrate, borate, bromide, camsylate, carbonate, chloride, clavulanate, citrate, dihydrochloride, edetate, edisylate, estolate, esylate, fumarate, gluceptate, gluconate, glutamate, glycollylarsanilate, hexyiresorcinate, hydrabamine, hydrobromide, hydrochloride, hydroxynaphthoate, iodide, isothionate, lactate, lactobionate, laurate, malate, maleate, mandelate, mesylate, methyibromide, methylnitrate, methylsulfate, mucate, napsylate, nitrate, N-methylglucamine ammonium salt, oleate, oxalate, pamoate (embonate), palmitate, pantothenate, phosphate/diphosphate, polygalacturonate, salicylate, stearate, sulfate, subacetate, succinate, tannate, tartrate, teoclate, tosylate, triethiodide and valerate. Furthermore, where the compounds of the disclosure carry an acidic moiety, suitable pharmaceutically acceptable salts thereof include, but are not limited to, salts derived from inorganic bases including aluminum, ammonium, calcium, copper, ferric, ferrous, lithium, magnesium, manganic, mangamous, potassium, sodium, zinc, and the like. Particularly preferred are the ammonium, calcium, magnesium, potassium, and sodium salts. Salts derived from pharmaceutically acceptable organic non-toxic bases include salts of primary, secondary, and tertiary amines, cyclic amines, and basic ion-exchange resins, such as arginine, betaine, caffeine, choline, N,N-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine, and the like.

The disclosed compounds that contain an acidic moiety may form salts with a variety of organic and inorganic bases. Exemplary basic salts include ammonium salts; alkali metal salts such as sodium, lithium, and potassium salts; alkaline earth metal salts such as calcium and magnesium salts; salts with organic bases (for example, organic amines) such as benzathines, dicyclohexylamines, hydrabamines (formed with N,N-bis(dihydroabietyl)ethylenediamine), N-methyl-D-glucamines, N-methyl-D-glucamides, t-butyl amines; and salts with amino acids such as arginine, lysine, and the like.

Basic nitrogen-containing groups may be quaternized with agents such as lower alkyl halides (e.g., methyl, ethyl, propyl, and butyl chlorides, bromides, and iodides), dialkyl sulfates (e.g., dimethyl, diethyl, dibutyl, and diamyl sulfates), long chain halides (e.g., decyl, lauryl, myristyl and stearyl chlorides, bromides and iodides), aralkyl halides (e.g., benzyl and phenethyl bromides), and others. Included are those esters and acyl groups known in the art for modifying the solubility or hydrolysis characteristics for use as sustained-release or prodrug formulations.

Solvates, and in particular, the hydrates of the compounds of the disclosure are included in the present disclosure as well.

The term “composition” as used herein is intended to encompass a product comprising the specified ingredients in the specified amounts, as well as any product which results, directly or indirectly, from combination of the specified ingredients in the specified amounts. Such a term in relation to a pharmaceutical composition is intended to encompass a product comprising the active ingredient(s), and the inert ingredient(s) that make up the carrier, as well as any product which results, directly or indirectly, from combination, complexation or aggregation of any two or more of the ingredients, or from dissociation of one or more of the ingredients, or from other types of reactions or interactions of one or more of the ingredients. Accordingly, the pharmaceutical compositions of the present disclosure encompass any composition made by admixing a compound of the present disclosure and a pharmaceutically acceptable carrier.

When a compound of the present disclosure is used contemporaneously with one or more other drugs, a pharmaceutical composition containing such other drugs in addition to the compound of the present disclosure is preferred. Accordingly, the pharmaceutical compositions of the present disclosure include those that also contain one or more other active ingredients, in addition to a compound of the present disclosure. The weight ratio of the compound of the present disclosure to the second active ingredient may be varied and will depend upon the effective dose of each ingredient. Generally, an effective dose of each will be used. Thus, for example, when a compound of the present disclosure is combined with another agent, the weight ratio of the compound of the present disclosure to the other agent will generally range from about 1000:1 to about 1:1000, preferably about 200:1 to about 1:200. Combinations of a compound of the present disclosure and other active ingredients will generally also be within the aforementioned range, but in each case, an effective dose of each active ingredient should be used. In such combinations the compound of the present disclosure and other active agents may be administered separately or in conjunction. In addition, the administration of one element may be prior to, concurrent to, or subsequent to the administration of other agent(s).

The terms “administration of” and or “administering a” compound should be understood to mean providing a compound of the disclosure or a prodrug of a compound of the disclosure to the individual in need of treatment.

The compositions may conveniently be presented in dosage unit form for the administration of the compounds of this disclosure and may be prepared by any of the methods well known in the art of pharmacy. All methods include the step of bringing the active ingredient into association with the carrier which constitutes one or more accessory ingredients. In general, the compositions are prepared by uniformly and intimately bringing the active ingredient into association with a liquid carrier or a finely divided solid carrier or both, and then, if necessary, shaping the product into the desired formulation. In the pharmaceutical composition the active object compound is included in an amount sufficient to produce the desired effect upon the process or condition of the hot flash.

Aqueous suspensions may contain the active materials in admixture with excipients suitable for the manufacture of aqueous suspensions. Such excipients are suspending agents, for example sodium carboxymethylcellulose, methylcellulose, hydroxy-propylmethylcellulose, sodium alginate, polyvinyl-pyrrolidone, gum tragacanth and gum acacia; dispersing or wetting agents may be a naturally-occurring phosphatide, for example lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl, p-hydroxybenzoate, one or more coloring agents, one or more flavoring agents, and one or more sweetening agents, such as sucrose or saccharin.

Oily suspensions may be formulated by suspending the active ingredient in a vegetable oil, for example arachis oil, olive oil, sesame oil or coconut oil, or in a mineral oil such as liquid paraffin. The oily suspensions may contain a thickening agent, for example beeswax, hard paraffin or cetyl alcohol. Sweetening agents such as those set forth above, and flavoring agents may be added to provide a palatable oral preparation. These compositions may be preserved by the addition of an anti-oxidant such as ascorbic acid.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified by those already mentioned above. Additional excipients, for example sweetening, flavoring and coloring agents, may also be present.

The pharmaceutical compositions of the disclosure may also be in the form of oil-in-water emulsions. The oily phase may be a vegetable oil, for example olive oil or arachis oil, or a mineral oil, for example liquid paraffin or mixtures of these. Suitable emulsifying agents may be naturally-occurring gums, for example gum acacia or gum tragacanth, naturally-occurring phosphatides, for example soy bean, lecithin, and esters or partial esters derived from fatty acids and hexitol anhydrides, for example sorbitan monooleate, and condensation products of the said partial esters with ethylene oxide, for example polyoxyethylene sorbitan monooleate. The emulsions may also contain sweetening and flavoring agents.

It will be understood, however, that the specific dose level and frequency of dosage for any particular patient may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the host undergoing therapy.

The term “organism” or “host” refers to any living entity comprised of at least one cell. As used herein, the term “host” includes mammals, and especially humans, in need of treatment.

The term “therapeutically effective amount” as used herein refers to that amount of the compound being administered that will relieve to some extent one or more of the symptoms of the condition or disorder being treated. In reference to treating hot flashes, a therapeutically effective amount refers to that amount that has the effect, among others, of (1) causing the host to which it is administered to be relieved from hot flash type symptoms, and/or (2) reducing the frequency or severity of a hot flash.

As used herein, a “pharmaceutically acceptable carrier” refers to a carrier or diluent that does not cause significant irritation to an organism and does not abrogate the biological activity and properties of the administered compound.

An “excipient” refers to an inert substance added to a composition to further facilitate administration of a compound. Examples of excipients include, without limitation, calcium carbonate, calcium phosphate, various sugars and types of starch, cellulose derivatives, gelatin, vegetable oils, and polyethylene glycols.

To the extent that the disclosed compounds, and salts thereof, may exist in their tautomeric form, all such tautomeric forms are contemplated herein as part of the present disclosure.

All stereoisomers of the present compounds, such as those which may exist due to asymmetric carbons on the various substituents, including enantiomeric forms (which may exist even in the absence of asymmetric carbons) and diastereomeric forms, are contemplated within the scope of this disclosure. Individual stereoisomers of the compounds of the disclosure may, for example, be substantially free of other isomers, or may be admixed, for example, as racemates or with all other, or other selected, stereoisomers. The chiral centers of the compounds of the present disclosure can have the S or R configuration as defined by the IUPAC 1974 Recommendations.

Unlike hormone replacement therapy, herbal treatments, oral medication, or any of the other options for hot flash sufferers, the disclosed composition is based on the physiology of a hot flash. There are no known or common side effects or risks associated with its use. It is believed that the disclosed composition and application thereof work to disrupt the brain's chemistry and essentially short-circuit thermostatic messages from the brain to the body. The disclosed composition is hormone-free and includes essential oils which work with the body and re-regulate the brain chemistry.

The disclosed composition generally comprises menthol in a solution which can be sprayed topically upon certain areas of the body. Menthol is an organic compound made synthetically or obtained from peppermint or other mint oils. It has local anesthetic and counterirritant qualities. Menthol also acts as a weak kappa Opioid receptor agonist. Menthol's ability to chemically trigger cold-sensitive TRPM8 receptors in the skin is responsible for the cooling sensation it provokes when applied to the skin.

The menthol is diluted and suspended in a medium which can be sprayed and applied topically. The menthol is typically suspended in an alcohol and water solution. In a particularly preferred embodiment, 10%-30% by weight menthol crystals are dissolved in 30%-60% alcohol SD-40, also known as SD Alcohol 40 or SDA-40. De-ionized water, 10%-60% by weight is then added to the mixture. To stabilize the suspension, a surfactant or emulsifier, may be incorporated into the composition as well. In a particularly preferred embodiment, Polysorbate 20, a polysorbate surfactant used as a detergent and emulsifier for stabilizing emulsions and suspensions, is added up to 10% by weight. Any essential oil may also be added up to 10% by weight in order to give the composition a pleasant smell. Peppermint essential oil is preferred as it also enhances the cooling effect of the composition.

A particularly preferred disclosed composition is prepared by dissolving 21% by weight menthol crystals into 48% by weight SDA Alcohol-40, stirring constantly until completely dissolved. Polysorbate 20 (3% by weight) and the peppermint essential oil (1.2% by weight) are combined with one another and stirred slowly for several minutes, typically five minutes. The peppermint essential oil/polysorbate 20 mixture is then slowly poured into the alcohol/menthol mixture, while stirring. This solution is clear, or stirred until clear. The de-ionized water (26.8% by weight) is then added to the mixture slowly while stirring. The solution will initially be cloudy, but will clear while stirring. If the solution does not clear entirely, additional alcohol is slowly added while stirring until the mixture clears.

As discussed above, the hot flash or night sweat begins when portions of the body's brain that control body temperature begin to react to various issues such as drop in hormone level, effect of chemotherapy, etc. The brain's insular cortex, which controls perceptions of heat, cold, pain and pleasure, begins to be activated. The core body temperature begins to rise in reaction to this. The temperature rises due to dilation of blood vessels and increased blood flow, which can result in dizziness and anxiety. The individual feels intensely hot, primarily in the upper portions of his or her body. The sweat glands activate and blood rushes to the extremities and face, neck and chest. The peripheral blood vessels dilate and heat shoots throughout the body, causing sweats.

When experiencing a hot flash or night sweat, the solution is topically applied to the body. More particularly, it has been found that when the composition is topically applied to the back of the neck, just below the hairline, an immediate relief of the sensation of heat, and a disruption of sweating and normalization of the body's temperature occurs.

In one embodiment, the composition is sprayed, such as through a hand-held spray bottle, onto the back of the neck just below the hairline. The high grade purified cosmetic alcohol evaporates nearly instantly, and thus serves as a vehicle to transport important ingredients to the skin's surface and then leave them there. Evaporation of the alcohol also has a cooling effect. Moreover, as discussed above, the menthol and peppermint oil also have cooling effects. It is believed that the menthol and/or peppermint oil trigger various nerve receptors and cells so as to send different signals along the brain's hypothalamus nerve cell pathway. The result is an immediate cessation of the signals to dilate blood vessels and increase blood flow.

Thus, in accordance with one embodiment, at first indication of a hot flash or night sweat, a spray bottle is held several inches away from the back of the neck near the base of the hairline. The composition is sprayed lightly on the back of the neck and used as needed. The use of the disclosed compositions is dependent upon the frequency of the hot flashes, although it is believed that the frequency of the hot flashes or night sweats will decrease with use of the spray over time. It is also believed that a benefit of the disclosed composition is the ability to stop the hot flash or night sweat from occurring at all with regular usage and appropriate timing between intervals. The elimination of night sweats and irregular sleep patterns as well as relief of headaches and irritability associated with menopausal symptoms are additional benefits.

Although several embodiments have been described in detail for purposes of illustration, various modifications may be made without departing from the scope and spirit of the invention as set forth in the following claims.

1. A chemical composition for reducing the effects of hot flashes or night sweats of the human body, comprising:
a diluted menthol suspended in a spray for topical application to the human body,
said diluted menthol comprising an alcohol and water solution.

2. The chemical composition of claim 1, wherein said diluted menthol comprises 10% to 30% by weight menthol crystals dissolved in 30% to 60% by weight of alcohol SD-40 and 10% to 60% by weight de-ionized water.

3. The chemical composition of claim 2, further including a stabilizer chosen from at least one of the following: a surfactant and an emulsifier.

4. The chemical composition of claim 3, wherein said surfactant comprises up to 10% by weight Polysorbate 20.

5. The chemical composition of claim 2, further comprising up to 10% peppermint essential oil.

6. A method of treating the effects of hot flashes or night sweats of the human body, comprising topically applying the chemical composition of claim 4 to the human body.

7. The method of claim 6, wherein topically applying the chemical composition comprises applying the chemical composition to the back of the neck of the human body.

8. A method of preparing a chemical composition for reducing the effects of hot flashes or night sweats of the human body, comprising
dissolving 21% by weight of menthol crystals into 48% by weight SDA Alcohol-40, and stirring constantly until completely dissolved,
combining 3% by weight polysorbate 20 and 1.2% by weight peppermint essential oil and stirring for several minutes,
pouring the peppermint essential oil and polysorbate 20 mixture into the alcohol and menthol mixture while stirring as may be necessary until clear, and
adding 26.8% by weight de-ionized water to the mixture while stirring until clear.
